(19) United States
(12) Patent Application Publication
(10) Pub. No.: US 2015/0174190 A1
(43) Pub. Date: Jun. 25, 2015

(54) Formulation Against General, Physical, And/Or Mental Fatigue

(71) Applicant: Hanan Polansky, Rochester, NY (US)

(72) Inventor: Hanan Polansky, Rochester, NY (US)

(21) Appl. No.: 14/139,893

(22) Filed: Dec. 24, 2013

(51) Int. Cl. A61K 36/82; A61K 31/353; A61K 33/04; A61K 36/54; A61K 36/484

(52) U.S. Cl. CPC A61K 36/82; A61K 36/54; A61K 36/484; A61K 33/04; A61K 31/353

(57) ABSTRACT

This patent presents a formulation that safely and effectively decreases the feeling of fatigue in treated individuals. Specifically, the natural formulation decreases the feeling of general fatigue, the feeling of physical fatigue, and the feeling of mental fatigue.

This non-provisional application is a continuation-in-part of, and claims the benefit of, U.S. non-provisional application Ser. No. 13/893,564 that was filed on 14 May 2013, entitled: Natural Product Against Fatigue, the entire disclosure of which is hereby incorporated by reference (hereafter “564 application). The 564 application claims the benefit of U.S. provisional application Ser. No. 61/646,910, filed on 15 May 2012, entitled: Natural Product for Increasing Levels of Energy, the entire disclosure of which is hereby incorporated by reference.

### BACKGROUND OF THE INVENTION

Studies have shown that one-third of the general population reports a feeling of fatigue, with 5% reporting a feeling of short-term fatigue (<6 months), and 31% reporting a feeling of long term or chronic fatigue (>6 months)¹. In the US alone, the feeling of fatigue is responsible for 10-15 million visits to the family doctor every year².

Studies associated the feeling of fatigue with an impaired physical and/or cognitive performance³.

Many diseases and conditions are associated with a feeling of fatigue (e.g., endocrine or metabolic problems, such as, Cushing's disease, kidney disease, electrolyte imbalances, diabetes, hypothyroidism, anemia, and liver disease, heart and lung conditions, such as pneumonia, arrhythmias, asthma, chronic obstructive pulmonary disease, valvular heart disease, coronary heart disease, and congestive heart failure, and other diseases such as cancer, anemia, multiple sclerosis, CFS sleeping problems, jet lag, sleep apnea, narcolepsy, insomnia, and reflux esophagitis, fibromyalgia, systemic lupus, rheumatoid arthritis, obesity, massive blood loss, weakened immune systems, chronic pain, and being overweight and underweight), and psychiatric disorders (e.g., stress, depression, and anxiety).

Many drugs are also associated with a feeling of fatigue (e.g., antihypertensives, β-adrenergic blockers, lipid-lowering agents, steroids, antihistamines, sedatives, and anti-anxiety, chemotherapy, radiotherapy, radiation therapy).

Fatigue is associated with many signs and symptoms. They can be physical, mental, or emotional in nature. The following is a list of some of these signs and symptoms: bloating, constipation, abdominal pain, diarrhea, nausea, painful, sore, or aching muscles, pain in lymph nodes, lack of interest, apathy, lack of motivation, drowsiness, short- or long-term tiredness, problems concentrating, hallucinations, dizziness, impaired physical and or mental coordination, headache, poor judgment, indecisiveness, irritability, lack of appetite, moodiness, weak immune system, long-term and short-term memory problems, language problems, sleepiness, sleeplessness, slow reaction time, slow reflexes, and vision problems.

Studies showed that one of the most important causes of a feeling of fatigue is an infection, specifically with a latent virus, such as the Epstein-Barr virus (EBV), human herpesvirus 6 (HHV-6), human herpesvirus 7 (HHV-7), herpes simplex virus (HSV), human immunodeficiency virus (HIV), hepatitis viruses, parvovirus B 19, and influenza virus^{4,5,6,7}.

### BRIEF SUMMARY OF THE INVENTION

This patent presents a formulation that safely and effectively decreases the feeling of fatigue in treated individuals. Specifically, the formulation decreases the feeling of general fatigue, the feeling of physical fatigue, and the feeling of mental fatigue.

### DETAILED DESCRIPTION OF THE INVENTION

Gene-Eden-VIR is a formulation designed to control the viral copy number in latently infected individuals, that is, asymptomatic individuals.

The following is the formulation of Gene-Eden-VIR: quercetin 100 mg, green tea extract 150 mg, cinnamon extract 50 mg, selenium 100 mcg, and licorice extract 25 mg.

The current invention centers on the fact that the Gene-Eden-VIR formulation decreases the feeling of fatigue in treated individuals, and more specifically, the feeling of general fatigue, the feeling of physical fatigue, and/or the feeling of mental fatigue.

### DEFINITIONS

Fatigue: Also means tiredness, lack of energy, weariness, exertion, lack of, exhaustion, yawning, drowsiness, shortness of breath, faintness, lack of ability, inability, sleepiness, laziness, lack or loss of motivation, procrastination, lethargy, and listlessness, weakness

Physical: Also means muscle, bodily, physiological, strength, animal

Mental: Also means psychological, psychiatric, cognitive, mind, thinking, concentration, attention, intellectual, calculation, processing, memory, perception, awareness, alertness, psychic, spiritual, subjective, brainy, clairvoyant, inner, subconscious, reasoning, rational, cerebral, visionary

### Examples

The following section reports the results of a clinical study that proved that the Gene-Eden-VIR formulation decreases the feeling of fatigue in treated individuals.

### Ethics Statement:

An informed consent was obtained from all subjects prior to conducting the phone interviews.

### Objective and Framework

We used this post marketing study to test the efficacy, safety, and optimal use of the Gene-Eden-VIR formulation on individuals suffering from fatigue. Specifically, we tested the effect of the Gene-Eden-VIR formulation on the general feeling of fatigue, the feeling of physical fatigue, and the feeling of mental fatigue.

### Treatment

Gene-Eden-VIR is a natural formulation. A capsule of the Gene-Eden-VIR formulation includes 100 mg of quercetin, 150 mg of green tea extract, 50 mg of cinnamon extract, 25 mg of licorice extract, and 100 mcg of selenium. The treatment included 1, 2, 3, or 4 capsules per day, and lasted 2 to 54 weeks.

### Questionnaire

As of today, there is no objective tool to measure fatigue. The attempts to objectively measure fatigue have failed. Currently, researchers and clinician assess fatigue assessments by using a number of subjective scales. We discovered that none of these tools are effective or relevant for our research. Therefore, we used a self-developed questionnaire called the Fatigue Natural Origin Treatment Clinical Questionnaire (F-NotCiq). The F-NotCiq questionnaire is a patient reported outcome (PRO) instrument. We successfully used a version of this tool in our previous researches. The purpose of a PRO instrument is to capture the patient's experience. In our current study, our main endpoint was the feeling of fatigue. Meaning, the objective of the study was to measure the effect of the treatment with Gene-Eden-VIR on the feeling of fatigue as reported by the treated participants.

The study collected the answers to the F-NotCiq instrument by phone interviews. We used two independent companies that specialized in outbound call services for performing the interviewers, one company from the US and one from Israel. The interviewers were blinded to the objective of the study. All interviews were recorded.

### Population

The study randomly selected participants from the Gene-Eden VIR customer database that includes all Gene-Eden-VIR current and past customers. The traditional response rate to phone interviews is 10-15%. Since the study was aiming to collect 100-150 interviews, the study used a computerized system to randomly create a call list of a 1000 customers. The final list of participants consisted of 100 Americans of both sexes, ages 20 to 66, infected with the HPV, EBV, HSV, HCMV, and HCV.

Since the objective of the study was to test the effect of the Gene-Eden-VIR formulation on the feeling of fatigue, the study excluded participants who reported no general feeling of fatigue, no feeling of physical fatigue, or no feeling of mental fatigue. That is, it excluded participants who reported a 7 point score on the pre-treatment question in these sections. Such score indicates that the participant does not suffer from the specific aspect of fatigue indicated by the question regardless of the treatment the participant actually received. This exclusion still preserved the intention to treat (ITT) principle.

At the start of the study, all participants reported a feeling of fatigue. Specifically, 98 reported a feeling of general fatigue, 90 reported a feeling of physical fatigue, and 79 reported a feeling of mental fatigue. Before treatment, 73 participants reported a general feeling of fatigue and a feeling of physical and mental fatigue.

The study considered participants, who stopped taking the Gene-Eden-VIR formulation for a month or more before data collection, as past users. All other participants were considered as present users.

### Controls

The Gene-Eden-VIR formulation post marketing study includes a pre-treatment concurrent control and an historical control. To create an historical control, we divided the original test group into two subgroups, present users and past users. Generally, an historical control is a separate group. However, since we did not have a separate group of non-users, we used the past users as a proxy for an historical control.

### Statistical Analysis

We tested the statistical difference between the score of ‘pre-treatment’, which is the numeric answer each participant used to describe his level of fatigue before the treatment started, to the score of ‘post-treatment’, which is the numeric answer each participant used to describe his level of fatigue after the treatment was completed. We also calculated the delta (Δ), that is, the difference in scores between the answers to the pre-treatment and post-treatment question. Then, we tested the statistical difference between the deltas. These tests were performed in a then-test model for both present and past users.

Statistical analysis was performed using a two-tail t-test assuming unequal variances.

The research defined the primary endpoint as a statistically significant increase in the score from pre-treatment to post-treatment on the raw answers and on the deltas.

### Results

The participants reported no side effects from taking the Gene-Eden-VIR formulation.

Following treatment with the Gene-Eden-VIR formulation, 72 out of the 98 participants (73.47%), who reported a feeling of general fatigue, reported a decrease in their fatigue, 56 out of the 90 (62.22%), who reported a feeling of physical fatigue, reported a decrease in their fatigue, and 36 out of the 79 (47.36%), who reported a feeling of mental fatigue, reported a decrease in their fatigue. Moreover, out of the 73 (73%) participants, who reported a feeling of both physical and mental fatigue, 62 (84.93%) reported a decrease in at least one of those types of fatigue [Table 2].

TABLE 2
F-NotCiq Summary

| Section | N of Participants* | |
|---|---|---|
| F - General fatigue | | |
| Pre-T general fatigue** | 98/100 | (98.00%) |
| Post-T decrease in general fatigue*** | 72/98 | (73.47%) |
| P - Physical fatigue | | |
| Pre-T physical fatigue | 90/100 | (90.00%) |
| Post-T decrease in physical fatigue | 56/90 | (62.22%) |
| M-Mental fatigue | | |
| Pre-T mental fatigue | 79/100 | (79.00%) |
| Post-T decrease in mental fatigue | 36/79 | (47.36%) |
| Pre-T fatigue | | |
| General, physical and mental fatigue | 73/100 | (73.00%) |
| Physical or mental fatigue and general fatigue | 93/100 | (93.00%) |
| Physical or mental fatigue | 95/100 | (95.00%) |
| Physical and general fatigue | 89/100 | (89.00%) |
| Mental and general fatigue | 77/100 | (77.00%) |
| Only general fatigue | 4/100 | (4.00%) |
| Post-T changes | | |
| Decrease in physical and/or mental fatigue | 62/73 | (84.93%) |
| Decrease in both physical and mental fatigue | 32/73 | (43.83%) |
| Decrease in general fatigue and in one type of | 54/73 | (73.97%) |
| fatigue | | |
| Decrease in general fatigue and physical fatigue | 51/89 | (57.30%) |
| Decrease in general fatigue and mental fatigue | 34/73 | (46.57%) |

*N of P is Number of Participants;
**Pre-T is Pre Treatment;
***Post-T is Post Treatment;

Following treatment with the Gene-Eden-VIR formulation, the participants reported a statistically significant decrease in every tested aspect of fatigue [Table 3].

TABLE 3
Aspects of fatigue, pre-treatment vs. post-treatment

| | | Pre-T | Post-T | |
|---|---|---|---|---|
| Questions | N of P^{#} | Score | Score | P Value |
| General fatigue | | | | |
| F1 - Level of energy | 88 | 4.65 | 5.74 | <0.001 |
| F2 - Tiredness | 85 | 4.14 | 5.36 | <0.001 |
| F3 - Ability to sleep | 82 | 4.44 | 5.23 | <0.001 |
| Physical fatigue | | | | |
| P1 - Strength | 70 | 4.77 | 5.49 | <0.001 |
| P2 - Ability to do your job | 43 | 4.95 | 5.51 | 0.05 |
| P3 - Ability to exercise | 50 | 4.63 | 5.58 | 0.001 |
| P4 - Ability to do housework | 47 | 4.89 | 5.53 | 0.018 |
| P5 - Ability to get out of bed | 55 | 4.42 | 5.36 | <0.001 |
| P6 - Stiffness | 63 | 4.51 | 5.13 | 0.013 |
| Mental fatigue | | | | |
| M1 - Attention span | 68 | 4.85 | 5.46 | 0.003 |
| M2 - Ability to form thoughts | 53 | 5.09 | 5.57 | 0.033 |
| M3 - Ability to concentrate | 60 | 4.80 | 5.47 | 0.005 |
| M4 - Ability to remember | 69 | 4.86 | 5.43 | 0.002 |

The statistical analysis was conducted using one score per participant.
^{#}N of P is Number of Participants, Pre-T is Pre-Treatment, Post-T is Post-Treatment.

To test for a duration effect, we compared the change (Δ) from pre-treatment to post-treatment in participants who took the Gene-Eden-VIR formulation for less then two months and those who took the Gene-Eden-VIR formulation for two months or more. The results showed that participants who took the Gene-Eden-VIR formulation for the longer period reported a larger decrease in their fatigue. Specifically, the participants reported a statistically significant decrease in their general feeling of fatigue, and in the their feeling of both physical and mental fatigue [Table 4].

TABLE 4
Duration of treatment

| | | Change (Δ) from | |
|---|---|---|---|
| Duration of Treatment | N of P* | Pre-T to Post-T | Statistics |
| General fatigue | | | |
| Less than 2 Months | 32 | 0.45 | P = 0.03, n = 65 |
| 2 Months or More | 33 | 0.84 | |
| Types of fatigue | | | |
| Less than 2 Months | 34 | 0.44 | P = 0.05, n = 70 |
| 2 Months or More | 36 | 0.76 | |

*Statistical analysis was performed using the change in scores from Pre-T to Post-T reported by present users only. The analysis used one score per participant. The score was equal to the average of the answers to questions F1-F3 for general fatigue, and questions P1-P6 and M1-M4 for types of fatigue.

To test for a possible interviewer bias, we compared the change (Δ) from pre-treatment to post-treatment collected by the American and the Israeli call centers. The change was tested for both general fatigue, and for the types of fatigue. In both cases, the difference between the answers was statistically not significant (p=0.79, n=254, for general fatigue, and p=0.49, n=582, for types of fatigue, Table 5). This means that although the centers included different interviewers from different cultures working at different times of day, Americans working during the day and Israelis working during the night, the answers were similar. Hence, the results showed no interviewer bias [Table 5].

TABLE 5
USA vs. Israel call centers.

| | USA | | Israel | | |
|---|---|---|---|---|---|
| | N of A | Score | N of A | Score | Statistics |
| General fatigue* | | | | | |
| Change (Δ) | 153 | 1.03 | 101 | 1.07 | P = 0.79, n = 254 |
| Types of fatigue** | | | | | |
| Change (Δ) | 323 | 0.69 | 259 | 0.63 | P = 0.49, n = 582 |

*Data may include up to three answers per participant.
**Data may include up to ten answers per participant.

To test for a possible selection bias, we compared the change (Δ) from pre-treatment to post-treatment by the past and present users of the Gene-Eden-VIR formulation.

The change was tested for both general fatigue, and for the types of fatigue. In both cases, the difference between the answers was statistically not significant (p=0.70, n=247, for general fatigue, p=0.15, n=568, for types of fatigue, Table 6). This means that, statistically, the answers by the present users are the same as the answers by the past users, and therefore, there was no selection bias [Table 6].

TABLE 6
Past vs. present users.

| | Present | | Past | | |
|---|---|---|---|---|---|
| | N of A | Score | N of A | Score | Statistics |
| General fatigue* | | | | | |
| Change (Δ) | 181 | 1.06 | 68 | 0.99 | P = 0.70, n = 247 |
| Types of fatigue** | | | | | |
| Change (Δ) | 411 | 0.61 | 157 | 0.77 | P = 0.15, n = 568 |

*Data may include up to three answers per participant.
**Data may include up to ten answers per participant.

An issue unique to natural products is the concern about the therapeutic consistency of marketed products. See discussion on this issue in the FDA guidelines for botanical New Drug Applications (NDA)⁹. To test the therapeutic consistency of the Gene-Eden-VIR formulation, we compared the two batches used by the participants. The capsules in these batches were produced at two different manufacturing sites, and completed about 10 months apart. The results showed that the answers given by the participants who used the capsules from Batch 1 were the same as those given by the participants who used the capsules from Batch 2 (p=0.62, n=256, for general fatigue, p=0.66, n=574, for types of fatigue Table 7). Hence, the results indicated that, although the Gene-Eden-VIR formulation includes natural ingredients, it has therapeutic consistency [Table 7].

TABLE 7
Batch 1 vs. Batch 2

| | Batch 1 | | Batch 2 | | |
|---|---|---|---|---|---|
| | N of A | Score | N of A | Score | Statistics |
| General fatigue* | | | | | |
| Change (Δ) | 147 | 1.06 | 109 | 0.98 | P = 0.62, n = 256 |
| Types of fatigue** | | | | | |
| Change (Δ) | 318 | 0.66 | 256 | 0.63 | P = 0.66, n = 574 |

*Data may include up to three answers per participant.
**Data may include up to ten answers per participant.

This post marketing clinical study showed that treatment with the Gene-Eden-VIR formulation safely decreased the feeling of fatigue in individuals who report a feeling of general fatigue, physical fatigue, and mental fatigue.

The results are consistent. We observed a statistically significant decrease in the feeling of general, physical, and mental fatigue.

The results also showed a duration effect. Participants treated for two months or more reported a larger decrease in their feeling of fatigue compared to those treated for less then two months.

The results in our study are consistent with results reported in other studies. For instance, Watt et al. 2012 showed that treatment with the antiviral drug Valganciclovir improved physical and cognitive performance in patients that suffered from fatigue. The study also showed that longer treatments were correlated with better results⁸.

The results are robust. They showed no interviewer bias, no selection bias, and therapeutic consistency of the Gene-Eden-VIR formulation under varying manufacturing conditions.

This post marketing clinical study does not include a placebo control, that is, it is not a double-blinded study. Placebo controlled studies are the gold standard in medical research in pre-marketing clinical studies. However, except in rare cases, post marketing studies do not use placebo controls. They use other controls recommended by the FDA.

The FDA guidance lists five types of controls for both pre-marketing and post marketing studies: 1) Placebo Concurrent Control, 2) Pre-treatment Concurrent Control, 3) Dose-response Concurrent Control, 4) Active (Positive) Concurrent Control, 5) External Control (Including Historical Control). The External Control “can be a group of patients treated at an earlier time (historical control)”⁹.

The Gene-Eden-VIR formulation post marketing study is a change from baseline study that includes a pre-treatment concurrent control and a proxy for an historical control.

The results are not likely to be a placebo effect. The current predominant and well proven theories on the placebo effect suggest that its main mechanisms are conditioned reflexes and patient expectations¹⁰. The Gene-Eden-VIR product literature does not mention the possibility of a change in the feeling of fatigue.

Hence, the participants in this study could not have been primed for, or expect the reported effects. This lack of conditioned reflexes and patient expectations minimizes the possibility of a placebo effect, and supports the possibility of a physiological effect.

All participants who started the study completed it; therefore, the study has no follow-up bias. It should be noted that although we tested for some biases, others are still possible, for instance, the non-responsive bias.

This study relies on patient reported outcomes (PROs). Past studies showed that PROs had a significant role in the development and evaluation of new medicines¹¹. According to the FDA, PROs are a valid and valuable source for measuring the efficacy of new drugs. They are reliable enough to warrant an approval of a label claim for a new drug. From the years 1997 to 2002, the FDA approved 23 new drugs based on results obtained in studies that used only PRO endpoints. They include six anti-migraine products (Amerge®, Ax-ert®), several anti-epileptics (Gabitril®, Keppra®), and a variety of other therapy classes (Tamiflu®, Relenza®)¹¹.

The FDA regards PROs as a valid and valuable source of data. The scientific community also believes that PROs are valid and useful. Many major journals published clinical studies that use patient reported outcomes. The trust of the FDA and the scientific community in PROs should convince the medical community, and specifically, doctors, to trust studies that use PROs when evaluating the benefits of new treatments.

A possible criticism of the F-NotCiq is that it uses a subjective scale, and therefore, may be producing biased results. However, the feeling of fatigue is essentially a subjective experience, and therefore, cannot be measured objectively.

The size of the study group is a major concern in clinical studies. A small group may fail to show a positive effect. In addition, a small group might misrepresent the diversity in the population. The standard principle for multivariate behavioral research is at least 10 patients for one endpoint, that is, for one dependent variable¹². This study included one endpoint, the change in the level of fatigue from pre-treatment to post-treatment. The population included 100 individuals. Hence, the size of the study group is adequate.

One might also question the reliability of the participants recall due to the long duration of the period under investigation (up to 54 weeks). This study used a “then-test” method. This method, which is also known as the retrospective pre-test-post-test design method, asks participants at the post-test period to think back to the pre-test period, and retrospectively rate their condition. The “response shift” is defined as the difference between the “pre-test” and the “then-test” ratings. Currently, the response shift is a well documented and extensively research phenomenon¹³. According to the literature on “response shifts,” participants may alter their internal standards, values, or conceptualization of their quality of life when experiencing changes in health states. The response shift can affect or distort the reported scores and undermine the credibility of the observed medical or psychosocial effects. Many studies reported that, after participants experience an improvement in their health, a then-test tends to show a decrease in the initial assessment of the original level of well being.

Since this clinical study uses the “then-test” method, we tested for a possible response shift by comparing the answers to the pre-treatment question at less then two months and at two months or more. The results showed a statistically significant increase in the pre-treatment score over time. The results indicated that the participants experience a response shift, however, in the opposite direction from what was expected. This response shift suggests that the participants do not tend to exaggerate, but tend to forget how bad their level of fatigue was before taking the Gene-Eden-VIR formulation. The tendency to forget adds support to the statistical significance of the results in this study.

To summarize, this post marketing clinical study showed that treatment with the Gene-Eden-VIR formulation safely decreased the feeling of fatigue in individuals reporting a feeling of general, physical, and/or mental fatigue. Therefore, the Gene-Eden-VIR formulation is a useful invention in the treatment of fatigue, where such fatigue is either reported by an individual, or implied by observing a certain disease or condition, or sign or symptom associated with fatigue.

### REFERENCES

1. Chalder T, Neeleman J, Reme S E, Power M, Wessely S. Factors associated with acute fatigue in primary care. Psychol Med. 2010 August; 40(8):1289-95
2. Chaudhuri A, Behan P O. Fatigue in neurological disorders. Lancet. 2004 Mar. 20; 363(9413):978-88
3. Shen J, Barbera J, Shapiro C M. Distinguishing sleepiness and fatigue: focus on definition and measurement. Sleep Med Rev. 2006 February; 10(1):63-76. Epub 2005 Dec. 22
4. Pantry S N, Medveczky M M, Arbuckle J H, Luka J, Montoya J G, Hu J, Renne R, Peterson D, Pritchett J C, Ablashi D V, Medveczky P G. Persistent human herpesvirus-6 infection in patients with an inherited form of the virus. J Med Virol. 2013 November; 85(11):1940-6
5. Karaivazoglou K, Iconomou G, Triantos C, Hyphantis T, Thomopoulos K, Lagadinou M, Gogos C, Labropoulou-Karatza C, Assimakopoulos K. Fatigue and depressive symptoms associated with chronic viral hepatitis patients. health-related quality of life (HRQOL). Ann Hepatol. 2010 October-December; 9(4):419-27
6. Barroso J, Hammill B G, Leserman J, Salahuddin N, Harmon J L, Pence B W. Physiological and psychosocial factors that predict HIV-related fatigue. AIDS Behav. 2010 December; 14(6):1415-27
7. Kondo K. Post-infectious fatigue. Japan Medical association Journal. 2006 49.1: 27.
8. Polansky H, Itzkovitz E. Gene-Eden-VIR Is Antiviral: Results of a Post Marketing Clinical Study. Pharmacology & Pharmacy. 2013 April: 1
9. Watt T, Oberfoell S, Balise R, Lunn M R, Kar A K, Merrihew L, Bhangoo M S, Montoya J G. “Response to valganciclovir in chronic fatigue syndrome patients with human herpesvirus 6 and Epstein-Barr virus IgG antibody titers”, J Med Virol., 2012 December; 84(12):1967-74
10. US Department of Health and Human Services, Food and Drug Administration, Center for Drug Evaluation and Research (CDER), “Guidance for Industry, E 10 Choice of Control Group and Related Issues in Clinical Trials,” 2001. http://www.fda.gov/downloads/RegulatoryInformation/Guidances/ucm129460.pdf
11. M. Breidert and K. Hofbauer, “Placebo: Misunderstandings and Prejudices,” Deutsches Ärzteblatt International, Vol. 106, No. 46, 2009, pp. 751-755
12. R. J. Willke, L. B. Burke and P. Erickson, “Measuring Treatment Impact: A Review of Patient-Reported Out-comes and Other Efficacy Endpoints in Approved Product Labels,” Controlled Clinical Trials, Vol. 25, No. 6, 2004, pp. 535-552
13. P. D. Harvey and R. S. Keefe, “Studies of Cognitive Change in Patients with Schizophrenia Following Novel Antipsychotic Treatment,” American Journal of Psychiatry, Vol. 158, No. 2, 2001, pp. 176-184
14. C. E. Schwartz, R. Bode, N. Repucci, et al., “The Clinical Significance of Adaptation to Changing Health: A Meta-Analysis of Response Shift,” Quality of Life Research, Vol. 15, No. 9, 2006, pp. 1533-1550

We claim the following:

1. A method for treating an animal or human subject, the method comprising the steps of:
a. Selecting an agent, wherein the agent consists of a green tea extract, quercetin, licorice extract, cinnamomum extract, and selenium;
b. Administering the agent to the subject to decrease the feeling of fatigue in said subject.

2. The method in claim 1, wherein said decrease in fatigue levels is indicated by an increase in energy levels.

3. The method in claim 1, wherein said feeling of fatigue is the feeling of physical fatigue.

4. The method in claim 1, wherein said feeling of fatigue is the feeling of mental fatigue.

5. The method in claim 1, wherein said subject is suffering from a disease or condition, or a sign or symptom associated with fatigue.
